(19) United States
(12) Patent Application Publication
(10) Pub. No.: US 2021/0353744 A1
(43) Pub. Date: Nov. 18, 2021

(54) ADMINISTRATION OF HOMOLOGOUS ADENOVIRAL VECTORS

(71) Applicant: Janssen Vaccines Prevention B.V., Leiden (NL)

(72) Inventors: Selina KHAN, Leiden (NL); Gerrit Christiaan SCHEPER, Amstelveen (NL); Roland Christian ZAHN, Rijnsburg (NL)

(21) Appl. No.: 17/317,580

(22) Filed: May 11, 2021

(30) Foreign Application Priority Data
May 12, 2020 (EP) 20174221.0

(51) Int. Cl. A61K 39/295; A61P 31/20

(52) U.S. Cl. CPC A61K 39/295; A61K 2039/54; A61P 31/20

(57) ABSTRACT

Compositions comprising a recombinant adenovirus of type Ad26 comprising a polynucleotide that encodes an antigen, kits of parts and methods of using these compositions for inducing an immune response in a subject are provided.

### SEQUENCE LISTING

Pursuant to 37 C.F.R. § 1.821(c) or (e), this application contains a sequence listing, which is contained on an ASCII text file entitled “Sequence Listing”

(SYT_3074_SequenceList_ST25.txt, created Tuesday, May 11, 2021, having a size of 20,811 bytes), which is herein incorporated by reference.

### FIELD OF THE INVENTION

This invention relates to the fields of medical microbiology, immunology and vaccines. In particular the invention relates to methods for inducing an immune response in a subject by an administration regimen for a recombinant adenovirus of type Ad26.

### BACKGROUND OF THE INVENTION

The genital tract mucosa is the major entry portal for most sexually transmitted diseases (STDs) of viral, bacterial, fungal, and parasitic origin; with over 100 million STD cases reported annually, this is a major worldwide health problem. It is well recognized that an effective vaccine should elicit long-term local mucosal immune responses to combat these infections.

For human papillomaviruses (HPV) infections, which cause cervical cancer, neither the magnitude nor the breadth of naturally occurring T cell responses detected in blood was a robust predictor of regression of preinvasive HPV disease of the cervix. However, both the magnitude and distribution of CD8+ T cell infiltrates in dysplastic cervical mucosa predict the chance of regression of HPV-induced lesions. Cervical lesions in which CD8+ T cell infiltrate the epithelial compartment were significantly more likely to undergo subsequent regression than lesions where such infiltration did not occur.

However, the genital tract is generally considered a poor inductive site of immune responses and induction of potent responses often requires the use of live infectious agents or a strong adjuvant and disruption of the mucosal lining. An efficient method for induction of strong local cellular immune responses in the genital tract that does not require mucosal disruption would be attractive in the field of therapeutic vaccines against HPV infections and other sexually transmitted infections.

Replication-deficient adenoviral vectors harbor the potential to induce strong humoral and cellular immune responses, either after a single or after repeated immunization. However, pre-existing immunity against certain adenoviruses (e.g. Ad5), either from natural exposure or from immunization, have been shown to dampen the immune responses after subsequent systemic immunization with Ad-based vectors. Using two vaccine components with different adenoviral type backbones (an approach known as ‘heterologous prime-boost’ regimen) avoids the negative effect of the immunity against the vector backbone that is induced by the first immunization. A disadvantage of this approach is the need for different products or vaccine components for the first and subsequent administration.

The immunogenicity of Adenovirus Types 26 (Ad26) and 35 (Ad35) vectors expressing a fusion of HPV16 E6 and E7 oncoproteins after intramuscular and/or intravaginal immunization has been evaluated in mice. The Ad26 and Ad35-based vectors were shown to transduce an intact cervicovaginal epithelium without the need of disruption. Intramuscular prime followed by intravaginal boost maximized the induction and trafficking of HPV-specific CD8+ T cells producing IFN-γ and TNF-α to the cervicovaginal tract. This prime-boost strategy targeting heterologous locations also induced circulating HPV-specific CD8+ T cell responses (Çuburu N, et al, 2018, Int J Cancer 142, 1467-1479). However, the immune response induced by the Ad26- and Ad35-based vectors upon immunizations via different administration routes in Çuburu et al were always tested in such a way that if the Ad26 vector was used for the prime immunization then Ad35 was used for the boost and vice versa (heterologous prime-boost), which has a disadvantage as described above. Moreover, prime-boost regimens with different adenovirus serotypes may not necessarily be predictive for prime-boost regimens with other serotypes, and the identity of the specific adenovirus serotype as well as the administration routes in an administration regimen may have an unpredictable impact on the immune response (see e.g. WO 2013/139916 for some examples of unpredictable differences between efficacy of different adenovirus serotypes). In a further study, Çuburu et al continue to use heterologous prime-boost regimens (using Ad5 vectors and HPV pseudoviruses as heterologous vectors) with combinations of intramuscular and intravaginal routes of administration, and conclude that their ‘study provides a rationale for heterologous prime-boost immunization using two unrelated nonreplicating viral vectors to maximize systemic and genital-resident memory T cell responses’ against vaccine antigens (Çuburu N, et al, 2019, J Immunol 2019; 202:1250-1264). Thus, those studies do not suggest vaccination regimens wherein the same vector would be administered at different body sites at different moments.

Despite the strong immune responses that can be elicited by intramuscular vaccination with adenoviral vectors, it is still unclear if such systemic immunizations, given as single injections or multiple doses will induce cellular immune responses that will be effective in the genital tract against already existing infections. Novel vaccine formulations and delivery strategies that induce more robust immunity in the genital mucosa need to be considered and tested.

An additional disadvantage having to administer multiple immunizations by intramuscular injections is the need for repeated visits to the doctor or clinic. In some settings, especially in low-income countries, limited access to health care could lead to large variations in the interval between first and subsequent immunizations. Or women may not have the possibility to return for the second immunization at all.

Accordingly, there remains a need in the art for treatment options to prevent or treat infection by pathogens in the genital mucosa. Furthermore, there is in particular a need for treatment options to induce a more robust immunity in the genital mucosa.

### SUMMARY OF THE INVENTION

In a first aspect, the invention provides a method for inducing an immune response in a subject comprising intramuscularly administering to the subject a recombinant adenovirus of type Ad26 comprising a polynucleotide that encodes an antigen (rAd26); and subsequently administering the rAd26 intravaginally to the subject. In certain embodiments the method is for inducing an immune response against an agent that causes a sexually transmitted disease (STD), such as HIV infection, HPV infection, HSV infection, chlamydia, gonorrhea, syphilis, or trichomoniasis.

In a second aspect, the invention provides a recombinant adenovirus of type Ad26 comprising a polynucleotide that encodes an antigen (rAd26) for use in inducing an immune response in a subject, wherein the rAd26 is for intramuscular administration, followed by intravaginal administration.

In a third aspect, the invention provides for a method for treating/and or preventing a viral infection in a subject comprising administering to the subject a recombinant adenovirus of type Ad26 comprising a polynucleotide that encodes an antigen of the virus that caused the viral infection or to which prevention is desired (rAd26); wherein the administration comprises intramuscular administration of the rAd26 followed by intravaginal administration of the rAd26.

In a fourth aspect, the method provides for a kit of parts comprising:
a first composition comprising a recombinant adenovirus of type Ad26 comprising a polynucleotide that encodes an antigen (rAd26); and

a second composition comprising the rAd26;

wherein the first composition is for use in intramuscular administration and the second composition is for use in intravaginal administration.

### BRIEF DESCRIPTION OF THE DRAWINGS

In order to better understand various exemplary embodiments, reference is made to the accompanying drawings, wherein:

FIG. 1A shows the experimental design of the HPV immunogenicity study;

FIG. 1B shows tetramer staining of antigen specific tissue resident memory CD8 T-cells in the cervicovaginal mucosal tissue;

FIG. 1C shows tetramer staining of antigen specific CD8 T-cells in the vaginal tract;

FIG. 2A shows experimental design of the immunogenicity study comparing short term regimen with long term regimen;

FIG. 2B shows tetramer staining of antigen specific tissue resident memory CD8 T-cells in the cervicovaginal mucosal tissue;

FIG. 2C shows serum levels of Ad26 neutralizing antibodies;

FIG. 2D shows tetramer staining of antigen specific CD8 T-cells in the spleen; and

FIG. 3 shows experimental design of the HIV specific CD8 T-cell immunogenicity study.

### DETAILED DESCRIPTION OF THE INVENTION

It has now surprisingly been found that a strong immune response in the genital mucosa can be achieved by homologous administration of an adenovirus of type Ad26 that comprises an antigen if the Ad26 is first administered intramuscularly and subsequently is administered intravaginally. Homologous administration of an adenovirus of type Ad26 is defined herein as administration of the same vector. That is, the adenovirus of type Ad26 comprising a polynucleotide that encodes an antigen in the first composition, is the same as the adenovirus of type Ad26 comprising a polynucleotide that encodes an antigen in the second composition. This has the advantage that only a single vector is needed, which makes administration easier, less error prone, and cheaper. Moreover, the use of only one instead of two vectors reduces the complexity, time, and costs for manufacturing. Furthermore, the route of intravaginal administration for the subsequent administration offers the possibility for self-administration, which would appear out of reach with repeated intramuscular injections that need to be administered by medically trained personnel.

Accordingly, in a first aspect, the invention provides for a method for inducing an immune response in a subject comprising: intramuscularly administering to the subject a recombinant adenovirus of type Ad26 comprising a polynucleotide that encodes an antigen (rAd26); and subsequently administering the same recombinant adenovirus (rAd26) intravaginally to the subject.

Unless defined otherwise, all technical and scientific terms used herein have the same meaning commonly understood to one of ordinary skill in the art to which this invention pertains. Otherwise, certain terms cited herein have the meanings as set in the specification.

All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Throughout this description and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The first and second administration according to the methods of the invention comprise administering the same recombinant adenovirus Ad26 (also referred to as recombinant adenoviral vector, rAd26). The preparation of recombinant adenoviral vectors is well known in the art. For example, preparation of rAd26 vectors is described, in WO 2007/104792 and in Abbink et al., 2007 Virology 81: 4654-63. Exemplary genome sequences of Ad26 are found in GenBank Accession EF 153474 and in SEQ ID NO:1 of WO 2007/104792.

As used herein, the term “adenovirus Ad26” is synonymous with “adenovirus of serotype Ad26” or “adenovirus of type Ad26”. A recombinant adenovirus Ad26 (“rAd26”) as used herein is an adenovirus Ad26 that includes a polynucleotide that encodes an antigen. Such polynucleotide can be introduced by standard genetic engineering methods known to the skilled person. Preferably the polynucleotide encoding the antigen is operably linked with a promoter that drives expression of the antigen, preferably in cells of the target organism to which the rAd26 is to be administered, such as a human. The antigen can be any antigen of interest to which an immune response is desired, and preferably is antigen expressed by a pathogen that can infect an organism such as a human via the genital mucosa.

A packaging cell line is typically used to produce sufficient amounts of adenovirus vectors of the invention. A packaging cell is a cell that comprises those genes that have been deleted or inactivated in a replication-defective vector, thus allowing the virus to replicate in the cell. Suitable cell lines include, for example, PER.C6, 911, 293, and E1-A549. Ad26 can for instance be generated in PER.C6 cells (Fallaux et al., 1998, Hum Gene Ther 9: 1909-17) by single homologous recombination and produced as previously described (WO 2007/104792; Abbink et al., 2007, J Virol 81: 4654-63). In preferred embodiments, the rAd26 in its genome has a replacement of Ad26 E4 orf6 by Ad5 E4 orf6, as for instance described in WO 03/104467 and in Abbink et al, supra.

In certain embodiments, a recombinant adenovirus according to the invention is deficient in at least one essential gene function of the E1 region, e.g. the Ela region and/or the E1b region, of the adenoviral genome that is required for viral replication. In certain embodiments, an adenoviral vector according to the invention is deficient in at least part of the non-essential E3 region. In certain embodiments, the vector is deficient in at least one essential gene function of the E1 region and at least part of the non-essential E3 region. The adenoviral vector can be “multiply deficient,” meaning that the adenoviral vector is deficient in one or more essential gene functions in each of two or more regions of the adenoviral genome. For example, the aforementioned E1-deficient or E1-, E3-deficient adenoviral vectors can be further deficient in at least one essential gene of the E4 region and/or at least one essential gene of the E2 region (e.g., the E2A region and/or E2B region).

The adenovirus used in the methods of the invention is a human adenovirus (HAdV, or AdHu; in the present invention a human adenovirus is meant if referred to Ad without indication of species, e.g. the brief notation “Ad26” means the same as HAdV26, which is human adenovirus serotype 26 or type 26).

To generate a strong immune response, prime-boost regimens are generally preferred. Therefore, in one embodiment of the invention, the first, intramuscular, administration of the adenovirus can be seen as a primer to prime the immune response and the subsequent, intravaginal, administration of the adenovirus can be seen as a booster to boost the immunization. In the present invention, the same recombinant Ad26 adenovirus is used thus the administration method can be seen as a homologous prime-boost administration regimen.

The present invention thus also relates to methods of inducing immune responses in a subject by priming and subsequently boosting the immune responses, with a combination of homologous vectors.

In the method according to the invention, the intravaginally administered rAd26 (i.e. “the booster”) is administered at least once but can optionally be administered multiple times. It is also possible to repeat the intramuscular administration, either prior to the first intravaginal administration, and/or subsequent thereto. Preferably however, the rAd26 is administered via intramuscular administration only once. It is also preferred to administer the rAd26 via the intravaginal route only once. Possibly, a late booster administration of the rAd26 via the intravaginal route, e.g. after at least 2, 3, 4, or 5 years, preferably at least 6, 7, 8, 9 or 10 years after the first intravaginal administration (which in turn was subsequent to an intramuscular administration) could be beneficial to maintain long-term immunogenicity against the antigen encoded in the rAd26.

The polynucleotide that encodes an antigen according to the invention is a heterologous polynucleotide, i.e. a polynucleotide that is not naturally present in adenovirus of type Ad26. The polynucleotide can be introduced into the rAd26 by standard molecular biology techniques.

The polynucleotide that encodes an antigen according to the invention is preferably capable of raising in a host a protective immune response against that antigen, e.g., induces an immune response against that antigen, and/or produces an immunity in (i.e., vaccinating) a subject, that protects the subject against the disease or infection associated with that antigen. The antigen can be of any foreign substance, for example a pathogen. For example, the antigen is a viral, parasitical, fungal, bacterial, protozoan or tumor antigen.

According to embodiments of the invention, “inducing an immune response” as used herein encompasses providing protective immunity and/or vaccinating a subject against an infection, for prophylactic purposes, or alternatively causing a desired immune response or effect in a subject in need thereof against an infection, for therapeutic purposes, i.e., therapeutic vaccination. “Inducing an immune response” also encompasses providing a therapeutic immunity for treating against a pathogenic agent. Typically, for prophylactic vaccination, compositions and vaccines are administered to subjects who are not already infected by a target pathogen, whereas for therapeutic vaccination, compositions and vaccines are administered to a subject already infected by a target pathogen. In a preferred method of the invention a therapeutic immune response is induced by intramuscular and subsequent intravaginal administration of the rAd26 as defined herein. One of skill will recognize that the immune response in the present invention is induced against an antigen of a pathogen (sometimes referred to herein as ‘target pathogen’) or disease as described herein, which antigen is encoded by the polynucleotide in the rAd26.

As used in the context of the invention, the terms “prevent”, “preventing”, and “prevention” refers to the prevention or reduction of the recurrence, onset, development or progression of diseases or infection, preferably a sexually transmitted disease as defined herein, or the prevention or reduction of the severity and/or duration of sexually transmitted disease or one or more symptoms thereof.

As used herein, the terms “treat”, “treating” and “treatment” refer to the reduction or amelioration of the progression, severity, and/or duration of a disease or infection, preferably a sexually transmitted disease as defined herein, and/or reduces or ameliorates one or more symptoms of the disease.

The immune response can be a cellular immune response and/or a humoral immune response. In a preferred embodiment of the invention, the immune response comprises a cellular immune response, more preferably a CD8+ T-cell response.

Examples of intramuscular administration comprise injection into the deltoid muscle of the arm, or vastus lateralis muscle of the thigh.

In the method according to the invention, the subsequent, e.g. second, administration of the rAd26 according to the invention is an intravaginal administration. Examples of intravaginal administration comprise topical administration for example topical administration by a viscous composition such as a gel. Gels may for instance comprise carboxymethylcellulose, polymer, or any other gel-forming or viscous substances, e.g. WO2017069793. In certain embodiments, pharmaceutical compositions described herein are administered intravaginally by a delivery vehicle, for example a capsule or a suppository. Suppositories for vaginal administration of the composition as defined herein can be prepared by mixing with a suitable nonirritating excipient such as cocoa butter and polyethylene glycols that are solid at room temperature but liquid at body temperature and will therefore melt in the vaginal tract and release the composition.

A subject as used herein preferably is a mammal, or a non-human-primate, or a human. Preferably, the subject is a human subject.

The rAd26 used in the invention may be present in a composition, e.g. a first composition for the intramuscular administration and a second composition for the intravaginal administration. The first and second compositions may be the same or different, e.g. they may optionally comprise different excipients tailored towards the intended administration route. The compositions, e.g. the first and second compositions, used according to the invention are preferably pharmaceutical compositions that may comprise any pharmaceutically acceptable excipient including a carrier, filler, preservative, solubilizer and/or diluent. Pharmaceutical compositions as described herein for use according to the invention may be in any form suitable for the intended method of administration, including, for example, a solution, a suspension, an emulsion or a gel. In a preferred embodiment, a composition as defined herein is administered in a solid form or in a liquid form. In the present context, the term “Pharmaceutically acceptable” means that the carrier or excipient, at the dosages and concentrations employed, will not cause any unwanted or harmful effects in the subjects to which they are administered. Such pharmaceutically acceptable excipients are well known in the art, and are extensively described in standard textbooks in the field. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, carboxymethylcellulose, ethanol and the like.

In some embodiments, the first and/or second compositions of the invention can further optionally comprise an adjuvant to enhance immune responses. The terms “adjuvant” and “immune stimulant” are used interchangeably herein, and are defined as one or more substances that cause stimulation of the immune system. In certain embodiments, the compositions do not comprise an adjuvant.

In certain embodiments, the subsequent, intravaginal, administration of the rAd26 is about one to ten weeks, e.g. two to eight weeks, after the first, intramuscular, administration of the rAd26 to the subject. In certain embodiments, the second composition is administered about one, two, three, four, five, six, eight, nine, or ten week(s) subsequent to administration of the first composition. Typically, shorter time intervals, e.g. 1, 2, 3, 4, 5, 6, 7, or 8 weeks, are practically preferred for expected improved compliance to the regimen.

In certain embodiments, the time interval between the intramuscular and intravaginal administration is at least 1 week, 2 weeks, 3 weeks, or at least 1, 2, 3, 4, 5, 6, 7, 8, 9, 10, 11, 12, or more months.

The antigen encoding polynucleotide according to the invention preferably encodes a viral, parasitical or bacterial antigenic protein or an immunogenic polypeptide thereof. The term “protein” or “polypeptide” refers to a molecule consisting of a chain of amino acids, without reference to a specific mode of action, size, 3-dimensional structure or origin. A “fragment” or “portion” of a protein may thus still be referred to as a “protein.” A protein as defined herein and as used in any method as defined herein may be an isolated protein. An “isolated protein” is used to refer to a protein which is no longer in its natural environment, for example in vitro or in a recombinant bacterial or animal host cell. The antigenic protein or immunogenic peptide can be any protein or peptide comprising an epitope (or antigenic determinant) In a preferred embodiment of the present invention, the polynucleotide in the rAd26 encodes a viral antigen, a parasitical or a bacterial antigen. Such antigens can be obtained by sequencing the genomes of the wild-type strains of the different viruses, parasites or bacteria, subcloning the nucleic acids encoding the antigenic determinants from such genomes, and cloning them into the adenoviral genomic sequence. Upon administration to a subject, the polypeptide encoded by the polynucleotide in the rAd26 according to the invention will be expressed in the subject, which will lead to an immune response towards the antigenic fragments that are present in the polypeptide.

“Amino acid sequence”: This refers to the order of amino acid residues of, or within a protein. In other words, the order of amino acids in a protein may be referred to as amino acid sequence.

“Nucleotide sequence”: This refers to the order of nucleotides of, or within a nucleic acid. In other words, the order of nucleotides in a nucleic acid may be referred to as nucleotide sequence.

Methods to insert heterologous coding sequences into an adenoviral genome are well known to the person skilled in the art. For example, methods for standard molecular biology techniques such as cloning of DNA, DNA and RNA isolation, Western blot analysis, RT-PCR and PCR amplification techniques are described in known textbooks and manuals. In a preferred embodiment, the polypeptide encoding the bacterial, parasitical or viral antigen according to the invention is codon optimized for expression in mammalian cells, preferably human cells. Codon-optimization is a technology widely applied in the art. Typically, the heterologous coding sequence is cloned into the E1 and/or the E3 region of the adenoviral genome, but alternatively it is also possible to clone it in a different region of the adenoviral genome.

The heterologous coding sequence can be under the control of (i.e., operably linked to) an adenovirus-derived promoter (e.g. Major Late Promoter) or can be under the control of a heterologous promoter. Non-limiting examples of suitable heterologous promoters include the immediate early promoter of CMV (CMV promoter) and the Rous Sarcoma Virus Long Terminal Repeat promoter (RSV promoter). Preferably, the promoter is located upstream of the heterologous gene of interest within an expression cassette. A non-limiting example of a CMV promoter sequence that can be used in an Ad26 vector to drive expression of the antigen is provided in SEQ ID NO: 24 of WO 2017/102929.

Preferably, the polynucleotide in the rAd26 encodes an antigenic protein or an immunogenic peptide derived from an agent that causes a sexually transmitted disease selected from the group consisting of HIV infection, HPV infection, HSV infection, chlamydia, gonorrhea, syphilis and trichomoniasis.

In a preferred embodiment, the polynucleotide encodes an antigenic protein or immunogenic peptide of human papillomavirus (HPV). Preferably, the antigenic protein is from HPV strain HPV type 16 (HPV16), HPV type 18 (HPV18), HPV type 31 (HPV31), HPV type 33 (HPV33), HPV type 35 (HPV35), HPV type 39 (HPV39), HPV type 45 (HPV45), HPV type 51 (HPV51), HPV type 52 (HPV52), HPV type 56 (HPV56), HPV type 58 (HPV58), HPV type 59 (HPV59), HPV type 68 (HPV68), HPV type 73 (HPV73). More preferably, the antigenic protein is from HPV16 and/or HPV18.

In certain embodiments, the polynucleotide encodes at least one epitope of an HPV E1 protein, for example an HPV16 E1 protein (for example a polynucleotide encoding the sequence of GenBank accession number: AAA46936.1) or an HPV18 E1 protein (for example a polynucleotide encoding the sequence of GenBank accession number: AAA99516.1).

In certain embodiments, the polynucleotide encodes at least one epitope of an HPV E2 protein, for example an HPV16 E2 protein or an HPV18 E2 protein. In certain embodiments, the E2 protein may be inactivated in for instance its transactivation and/or DNA binding domain, e.g. by deletion, mutation or by structural rearrangement of different parts of the protein). In certain embodiments, E2 has mutations in the transactivation domain, in other embodiments E2 has mutations in the DNA binding domain, and in further embodiments E2 has mutations in both the transactivation domain and in the DNA binding domain. In yet another alternative embodiment, the E2 polypeptide is divided in fragments which are reordered (shuffled), to abrogate E2 activity while maintaining the E2 epitopes for immunogenicity. In certain embodiments, the E2 protein is a wild-type E2 protein. In certain other embodiments, the E2 protein has a deletion or one or more mutations in its DNA binding domain (as compared to a wild type E2 protein).

In certain embodiments, the polynucleotide encodes at least one epitope of an HPV E6 protein, for example an HPV16 E6 protein or an HPV18 E6 protein. The HPV E6 protein may contain mutations and/or re-arrangements to delete its oncogenic potential, as previously described in the art.

In certain embodiments, the polynucleotide encodes at least one epitope of an HPV E7 protein, for example an HPV16 E7 protein or an HPV18 E7 protein. The HPV E7 protein may contain mutations and/or re-arrangements to delete its oncogenic potential, as previously described in the art.

In preferred embodiments of the invention, the polynucleotide encodes epitopes of both E6 and E7 of HPV, preferably epitopes of E6 and E7 of HPV16 and/or HPV18. In certain embodiments the polynucleotide encodes a fusion protein that comprises virtually the complete E6 and E7 amino acid sequences of HPV16 in the form of fragments that are re-ordered and partly overlapping such that (essentially) all T-cell epitopes of the HPV16 E6 and E7 protein are present, such as for instance described in detail in WO 2017/029360. In certain embodiments, the polynucleotide encoding the epitopes of both E6 and E7 further comprises at least one epitope of the E2 protein of HPV, such as for instance described in detail in WO 2017/029360. Some non-limiting examples of HPV antigens that can be used in the instant invention are those that have been set forth in WO 2017/029360, such as HPV16-E6E7SH (SEQ ID NO: 1 of WO 2017/029360), HPV16-E2E6E7SH (SEQ ID NO: 3 of WO 2017/029360), HPV16-E6E7E2SH (SEQ ID NO: 5 of WO 2017/029360), HPV18-E6E7SH (SEQ ID NO: 20 of WO 2017/029360), or HPV18-E2E6E7SH (SEQ ID NO: 22 of WO 2017/029360). Examples of suitable nucleic acid sequences encoding these antigens were also provided in WO 2017/029360, for instance as SEQ ID NO: 2 therein (encoding HPV16-E6E7SH), SEQ ID NO: 4 therein (encoding HPV16-E2E6E7SH), SEQ ID NO: 6 therein (encoding HPV16-E6E7E2SH), SEQ ID NO: 21 therein (encoding HPV18-E6E7SH) and SEQ ID NO: 23 therein (encoding HPV18-E2E6E7SH).

Other examples of HPV antigen sequences are available to the skilled person from public databases, such as the GenBank sequence database provided by the National Center for Biotechnology Information (NCBI) (“GenBank”).

Preferably, the polynucleotide encodes an HPV E6 protein or antigenic part thereof and/or an HPV E7 protein or antigenic part thereof. Even more preferably the polynucleotide encodes epitopes of both HPV E6 and E7 proteins. In certain embodiments the polynucleotide comprises or consists of SEQ ID NO: 1. In certain embodiments, the polynucleotide encodes a protein comprising or consisting of SEQ ID NO: 2.

In another preferred embodiment, the polynucleotide encodes an antigenic protein or immunogenic peptide of human immunodeficiency virus (HIV), preferably HIV-1. In one embodiment, the antigenic protein is or is derived from HIV Group Antigen (Gag), Polymerase (Pol), and/or Envelope (Env) proteins, or an antigenic part thereof. In one embodiment, the antigen is a “mosaic” antigen derived from HIV-1 Gag, Pol and/or Env antigens. Such mosaic antigens have been described by others and developed in an attempt to provide maximal coverage of potential T-cell epitopes (e.g., Barouch et al, Nat Med 2010, 16: 319-323). The mosaic antigens are similar in length and domain structure to wild-type, naturally occurring HIV-1 antigens. For example, mosaic HIV antigens described and used in vaccines include those described in Barouch et al, supra, and for instance in WO 2010/059732, or in WO 2017/102929. Non-limiting examples of suitable mosaic HIV antigens that can be used in the instant invention include one or more of: (i) mosaic Gag antigen sequences as set forth in WO 2017/102929, such as those having an amino acid sequence as set forth in SEQ ID NO: 1 (“mos1.Gag”) or SEQ ID NO: 2 (“mos2.Gag”) of WO 2017/102929; (ii) mosaic Pol antigen sequences as set forth in WO 2017/102929, such as those having an amino acid sequence as set forth in SEQ ID NO: 3 (“mos1.Pol”) or SEQ ID NO: 4 (“mos2.Pol”) of WO 2017/102929; (iii) mosaic Env antigen sequences as set forth in WO 2017/102929, such as those having an amino acid sequence as set forth in SEQ ID NO: 5 (“mos1.Env”) or SEQ ID NO: 18 (“mos2S.Env”) of WO 2017/102929; or fusions thereof, such as mos1.GagPol (SEQ ID NO: 28 in WO 2017/102929) or mos2.GagPol (SEQ ID NO: 29 in WO 2017/102929). Examples of suitable nucleic acid sequences encoding these antigens were also provided in WO 2017/102929, for instance as SEQ ID NO: 20 (encoding mos1.GagPol), SEQ ID NO: 21 (encoding mos2.GagPol), SEQ ID NO: 22 (encoding mos1.Env), and SEQ ID NO: 23 (encoding mos2S.Env) therein.

In one preferred embodiment of the invention, the polynucleotide encodes the fusion protein of Gag and Pol (e.g. “mos1.GagPol” or “mos2.GagPol”). In certain embodiments the polynucleotide comprises or consists of SEQ ID NO: 3. In certain embodiments, the polynucleotide encodes a protein comprising or consisting of SEQ ID NO: 4.

In another preferred embodiment of the invention, the polynucleotide encodes an Env polypeptide, e.g. mos1.Env or mos2S.Env. In certain embodiments, a combination of Ad26 vectors is used, wherein each Ad26 vector comprises a polynucleotide encoding an antigen as indicated above, and the combination of Ad26 vectors together encodes a combination of the antigens as indicated above, for instance a combination of (i) mos1.GagPol, (ii) mos2.GagPol, (iii) mos1.Env, and (iv) mos2S.Env. Such combinations of vectors can be mixed in a single composition (see e.g. WO 2017/102929).

Other examples of HIV Gag, Pol, Env antigen sequences, or other HIV antigen sequences such as Nef, Tat, Rev, Vif, Vpr, or Vpu, are available to the skilled person from public databases, such as GenBank. Many different variants of HIV antigens have been described and could be used in the invention. Another non-limiting example would be the HIV T-cell immunogens described in WO 2013/110818.

In certain embodiments, further components can be administered to a subject to which the vectors are administered according to the invention, e.g. by additionally administering isolated HIV Env protein antigen, such as gp140 protein (see e.g. WO 2017/102929, for instance one or both proteins having amino acids 30-708 of SEQ ID NO: 7 and/or amino acids 30-724 of SEQ ID NO: 36 of WO 2017/102929).

In certain embodiments, the polynucleotide encodes an antigenic protein or immunogenic peptide of herpes simplex virus (HSV), preferably HSV-2. In one embodiment, the antigenic protein is or is derived from an HSV protein selected from gH, gL, gM, gB, gC, gK, gE, gD ICP27, ICP47, ICP4, ICP36, VP22, RR2, UL19, UL47, VP11/12, or VP13/14.

In certain embodiments, the antigen encoding polynucleotide encodes an antigenic protein or immunogenic peptide of Chlamydia trachomatis. In one embodiment, the antigenic protein is or is derived from a Chlamydia trachomatis protein selected from PepA, Lcr, ArtJ, DnaK, CT398 OmpH-like, L7/L12, OmcA, AtoS, Eno, HtrA, MurG, MOMP, PmpD, MIP and Pgp3.

In certain embodiments, the polynucleotide encodes an antigenic protein or immunogenic peptide of Neisseria gonorrhoeae. In one embodiment, the antigenic protein is or is derived from a Neisseria gonorrhoeae protein selected from NGO0416, NGO0690, NGO0948, NGO1043, NGO1215, NGO1701, BamA, LptD, TamA, NGO2054, NGO2139, Lectin, Ag473, Omp85, and FrpB.

In certain embodiments, the polynucleotide encodes an antigenic protein or immunogenic peptide of Treponema pallidum. In one embodiment, the antigenic protein is or is derived from a Treponema pallidum protein selected from Tp92, Gpd, TprK and Tp0435, Tp0751 (Pallilysin), Flagellin, and 47-kilodalton integral membrane lipoprotein.

In certain embodiments, the polynucleotide encodes an antigenic protein or immunogenic peptide of Trichomonas vaginalis. In one embodiment, the antigenic protein is or is derived from a Trichomonas vaginalis protein selected from □-actinin or N-terminal enolases (Gen eIDs TVAG_329460, TVAG_043500, TVAG_464170, TVAG_358110, TVAG_263740, TVAG_487600, TVAG_170370, TVAG_2820).

Generally, sequences of proteins, such as those indicated above and other proteins, e.g. sequences of HSV, Chlamydia trachomatis, Neisseria gonorrhoeae, Treponema pallidum, and Trichomonas vaginalis proteins, are available to the skilled person in public databases, such as the GenBank sequence database provided by the National Center for of technology Information (NCBI).

In one embodiment, the method according to the invention is for inducing an immune response against a sexually transmitted disease (STD). A sexually transmitted disease, sometimes referred to sexually transmitted infections (STIs) or venereal diseases (VD), are diseases that are transmitted by sexual contact and are caused by microorganisms that survive on the skin or mucus membranes of the genital area; or transmitted via semen, vaginal secretions, or blood during intercourse. Some STDs can alternatively spread through the use of unsterilized drug needles, from mother to infant during childbirth or breast-feeding, and blood transfusions. Commonly known STDs include AIDS/HIV-infection, chlamydia, genital herpes, gonorrhea, HPV-infection-caused warts or cancer, trichomoniasis, and syphilis.

Accordingly, in certain embodiments, in the method according to the invention, the immune response is induced against an STD that is selected from the group consisting of HIV infection, HSV infection (for example genital herpes), HPV infection-induced diseases, chlamydia, gonorrhea, trichomoniasis and syphilis.

In one embodiment, the invention comprises a method of inducing an immune response against HIV. Human immunodeficiency virus infection and acquired immune deficiency syndrome (HIV/AIDS) is a spectrum of conditions caused by infection with the human immunodeficiency virus (HIV). HIV is spread primarily by unprotected sex (including anal and oral sex), contaminated blood transfusions, hypodermic needles, and from mother to child during pregnancy, delivery, or breastfeeding.

In one embodiment, the invention comprises a method of inducing an immune response against human papillomavirus (HPV). HPV infection is an infection caused by the human papillomavirus (HPV). Diverse types of HPV exist (over 120 types have been identified and are referred to by number), and generally for each type that needs to be covered by a vaccine, type-specific antigens may need to be incorporated in the vaccine, although for certain antigens some cross-reactivity might exist. Types 16, 18, 31, 33, 35, 39, 45, 51, 52, 56, 58, 59, 68, 73, and 82 are carcinogenic “high-risk” sexually transmitted HPVs and infection with these types may inter alia lead to the development of cervical intraepithelial neoplasia (CIN), vulvar intraepithelial neoplasia (VIN), vaginal intraepithelial neoplasia (VaIN), penile intraepithelial neoplasia (PIN), and/or anal intraepithelial neoplasia (AIN), as well as to cancer of the cervix, vulva, vagina, penis, anus, mouth, or throat.

In one embodiment, the invention comprises a method of inducing an immune response against HSV. HSV is classified into two types, HSV-1 and HSV-2. Infection by HSV can for instance cause the STD genital herpes, an infection by the herpes simplex virus (HSV) of the genitals.

In one embodiment, the invention comprises a method of inducing an immune response against Chlamydia trachomatis. Infection by Chlamydia trachomatis can cause the STD chlamydia, and this disease can manifest in a number of ways including: trachoma, lymphogranuloma venereum, nongonococcal urethritis, cervicitis, salpingitis, pelvic inflammatory disease.

In one embodiment, the invention comprises a method of inducing an immune response against Neisseria gonorrhoeae. Infection by the bacterium Neisseria gonorrhoeae can cause the STD gonorrhea. Infection may involve the genitals, mouth, and/or rectum.

In one embodiment, the invention comprises a method of inducing an immune response against Treponema pallidum. A sexually transmitted infection caused by the bacterium Treponema pallidum subspecies pallidum is known as syphilis.

In one embodiment, the invention comprises a method of inducing an immune response against Trichomonas vaginalis. A sexually transmitted infection caused by the parasite Trichomonas vaginalis is known as trichomoniasis.

In a further aspect, the invention provides for a recombinant adenovirus of type Ad26 comprising a polynucleotide that encodes an antigen (rAd26) for use in inducing an immune response in a subject, wherein the rAd26 is for intramuscular administration, followed by intravaginal administration. In certain embodiments, the rAd26 is for use in inducing an immune response against a bacterial, parasitical or viral antigen or antigenic fragment thereof as described herein. In certain embodiments, the rAd26 is for use in inducing an immune response against a STD as described herein.

In a preferred embodiment, the rAd26 is for use in inducing an immune response against HPV or an HPV infection and the polynucleotide as described herein encodes an HPV E6 protein or antigenic part thereof and/or an HPV E7 protein or antigenic part thereof. Preferably the polynucleotide encodes epitopes of both HPV E6 and E7 proteins.

In another preferred embodiment, the rAd26 is for use in inducing an immune response against HIV and the polynucleotide as described herein encodes an HIV Env, Gag or Pol protein or an antigenic part thereof.

In a further aspect, the invention provides for a method for treating and/or preventing a viral infection in a subject comprising administering to the subject a recombinant adenovirus of type Ad26 that comprises a polynucleotide that encodes an antigen of the virus of which the infection is to be treated and/or prevented (rAd26), wherein the administration to the subject comprises intramuscular administration of the rAd26 followed by intravaginal administration of the rAd26.

In certain embodiments, the administration of rAd26 to a subject according to methods of the invention comprises the administration of an effective amount of one or more compositions comprising the rAd26. As used herein, “an effective amount” or “immunologically effective amount” means an amount of a composition sufficient to induce a desired immune effect or immune response in a subject in need thereof. In one embodiment, an effective amount means an amount sufficient to induce an immune response in a subject in need thereof. In another embodiment, an effective amount means an amount sufficient to produce immunity in a subject in need thereof, e.g., provide a protective effect against a disease such as a viral infection. An effective amount can vary depending upon a variety of factors, such as the physical condition of the subject, age, weight, health, etc.; the particular application, whether inducing immune response or providing protective immunity; the specific recombinant vector administered; the immunogen or antigenic polypeptide encoded by the recombinant vector administered; the specific antigenic polypeptide administered; and the particular disease, e.g., viral infection, for which immunity is desired. An effective amount can readily be determined by one of ordinary skill in the art in view of the present disclosure. The total dose of the vaccine active component provided to a subject during one administration can be varied as is known to the skilled practitioner, and for adenovirus such as rAd26 is generally between 1×10⁷ viral particles (vp) and 1×10¹² vp, preferably between 1×10⁹ vp and 1×10¹¹ vp, for instance between 5×10⁹ vp and 5×10¹⁰ vp.

In yet a further aspect, the present invention provides for a kit of parts comprising a first composition comprising a recombinant adenovirus of type Ad26 as described herein and a second composition of type Ad26 as described herein. In particular embodiments, the kit of parts comprises a first composition comprising a recombinant adenovirus of type Ad26 comprising a polynucleotide that encodes an antigen (rAd26); and a second composition comprising the rAd26; wherein the first composition is for use in intramuscular administration and the second composition is for use in intravaginal administration.

The kit of parts is preferably for a use in inducing an immune response, preferably an immune response against a disease, infection or antigen as defined herein.

Optionally, the kit of parts further comprises a leaflet. The leaflet may comprise instructions for use. In addition, or alternatively, the leaflet may be at least one of a patient information leaflet and a Summary of Product Characteristics (an SmPC).

In a first aspect, the invention thus provides a method for inducing an immune response in a subject comprising intramuscularly administering to the subject a recombinant adenovirus of type Ad26 comprising a polynucleotide that encodes an antigen (rAd26); and subsequently administering the rAd26 intravaginally to the subject.

In a second aspect, the invention provides a recombinant adenovirus of type Ad26 comprising a polynucleotide that encodes an antigen (rAd26) for use in inducing an immune response in a subject, wherein the rAd26 is for intramuscular administration and subsequent intravaginal administration to the subject.

In a third aspect, the invention provides a use of a recombinant adenovirus of type Ad26 comprising a polynucleotide that encodes an antigen (rAd26) for the manufacture of a medicament for inducing an immune response, wherein the rAd26 is for intramuscular administration and subsequent intravaginal administration to the subject.

In a fourth aspect, the invention provides a kit of parts that comprises a first composition comprising a recombinant adenovirus of type Ad26 comprising a polynucleotide that encodes an antigen (rAd26); and a second composition comprising the rAd26; wherein the first composition is for use in intramuscular administration and the second composition is for use in intravaginal administration.

In a fifth aspect, the invention provides a method for treating/and or preventing a viral infection in a subject comprising administering to the subject a recombinant adenovirus of type Ad26 comprising a polynucleotide that encodes an antigen of the virus that caused the viral infection or to which prevention is desired (rAd26); wherein the administration comprises intramuscular administration of the rAd26 followed by intravaginal administration of the rAd26.

It will be clear to the skilled person that the description provided above of elements regarding the first aspect, such as origin and identity of antigens and/or polynucleotides, rAd26, administration routes, pharmaceutical compositions, timing of administration, subjects, diseases to be treated or prevented, etc, are equally applicable to those elements in the second, third, fourth, and fifth aspects.

Various publications, articles and patents are cited or described in the background and throughout the specification. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

### Description of the Sequences

TABLE 1
Sequences

| | | SEQ |
|---|---|---|
| Description | SEQUENCE | ID NO. |
| HPV16.E6E7 | ATGCACCAGAAACGGACCGCCATGTTCCAGGACCCCCAGGAACGGCCCAGAAAGC | 1 |
| (nucleotide | TGCCCCAGCTGTGCACCGAGCTGCAGACCACCATCCACGACATCATCCTGGAATG | |
| sequence) | CGTGTACTGCAAGCAGCAGCTCCTGCGGAGAGAGGTGTACGACTTCGCCTTCCGG | |
| | GACCTGTGTATCGTGTACCGGGACGGCAACCCCTACGCCGTGTGCGACAAGTGCC | |
| | TGAAGTTCTACAGCAAGATCAGCGAGTACCGGCACTACTGCTACAGCCTGTACGG | |
| | CACCACCCTGGAACAGCAGTACAACAAGCCCCTGTGCGACCTGCTGATCCGGTGC | |
| | ATCAACTGCCAGAAACCCCTGTGCCCCGAGGAAAAGCAGCGGCACCTGGACAAGA | |
| | AGCAGCGGTTCCACAACATCCGGGGCAGATGGACAGGCCGGTGCATGAGCTGCTG | |
| | CAGAAGCAGCCGGACCAGACGGGAAACCCAGCTGATGCACGGCGACACCCCCACC | |
| | CTGCACGAGTACATGCTGGACCTGCAGCCCGAGACAACCGACCTGTACTGCTACG | |
| | AGCAGCTGAACGACAGCAGCGAGGAAGAGGACGAGATCGACGGCCCTGCTGGACA | |
| | GGCCGAGCCTGATAGAGCCCACTACAATATCGTGACCTTCTGCTGCAAGTGCGAC | |
| | AGCACCCTGCGGCTGTGCGTGCAGAGCACCCACGTGGACATCCGGACCCTGGAAG | |
| | ATCTGCTGATGGGCACCCTGGGCATCGTGTGCCCCATCTGCTCCCAGAAACCCTG | |
| | ATAA | |
| HPV16.E6E7 | MHQKRTAMFQDPQERPRKLPQLCTELQTTIHDIILECVYCKQQLLRREVYDFAFR | 2 |
| (amino acid | DLCIVYRDGNPYAVCDKCLKFYSKISEYRHYCYSLYGTTLEQQYNKPLCDLLIRC | |
| sequence) | INCQKPLCPEEKQRHLDKKQRFHNIRGRWTGRCMSCCRSSRTRRETQLMHGDTPT | |
| | LHEYMLDLQPETTDLYCYEQLNDSSEEEDEIDGPAGQAEPDRAHYNIVTFCCKCD | |
| | STLRLCVQSTHVDIRTLEDLLMGTL | |
| Mos1.Gag- | ATGGGAGCCAGAGCCAGCGTGCTGTCCGGAGGGGAGCTGGACCGCTGGGAGAAGA | 3 |
| Pol. | TCAGGCTGAGGCCTGGAGGGAAGAAGAAGTACAGGCTGAAGCACATCGTGTGGGC | |
| (nucleotide | CAGCAGAGAGCTGGAACGGTTTGCCGTGAACCCTGGCCTGCTGGAAACCAGCGAG | |
| sequence) | GGCTGTAGGCAGATTCTGGGACAGCTGCAGCCCAGCCTGCAGACAGGCAGCGAGG | |
| | AACTGCGGAGCCTGTACAACACCGTGGCCACCCTGTACTGCGTGCACCAGCGGAT | |
| | CGAGATCAAGGACACCAAAGAAGCCCTGGAAAAGATCGAGGAAGAGCAGAACAAG | |
| | AGCAAGAAGAAAGCCCAGCAGGCTGCCGCTGACACAGGCAACAGCAGCCAGGTGT | |
| | CCCAGAACTACCCCATCGTGCAGAACATCCAGGGACAGATGGTGCACCAGGCCAT | |
| | CAGCCCTCGGACCCTGAACGCCTGGGTGAAGGTGGTGGAGGAAAAGGCCTTCAGC | |
| | CCTGAGGTGATCCCCATGTTCTCTGCCCTGAGCGAGGGAGCCACACCCCAGGACC | |
| | TGAACACCATGCTGAACACCGTGGGAGGGCACCAGGCTGCCATGCAGATGCTGAA | |
| | AGAGACAATCAACGAGGAAGCTGCCGAGTGGGACAGGGTCCACCCAGTGCACGCT | |
| | GGACCTATCGCTCCTGGCCAGATGAGAGAGCCCAGAGGCAGCGATATTGCTGGCA | |
| | CCACCTCCACACTGCAGGAACAGATCGGCTGGATGACCAACAACCCTCCCATCCC | |
| | TGTGGGAGAGATCTACAAGCGGTGGATCATTCTGGGACTGAACAAGATCGTGCGG | |
| | ATGTACAGCCCTGTGAGCATCCTGGACATCAGGCAGGGACCCAAAGAGCCCTTCA | |
| | GGGACTACGTGGACCGGTTCTACAAGACCCTGAGAGCCGAGCAGGCCAGCCAGGA | |
| | CGTGAAGAACTGGATGACCGAGACACTGCTGGTGCAGAACGCCAACCCTGACTGC | |
| | AAGACCATCCTGAAAGCCCTGGGACCTGCTGCCACCCTGGAAGAGATGATGACAG | |
| | CCTGCCAGGGAGTGGGAGGACCTGGCCACAAGGCCAGGGTGCTGGCCGAGGCCAT | |
| | GAGCCAGGTGACCAACTCTGCCACCATCATGATGCAGAGAGGCAACTTCCGGAAC | |
| | CAGAGAAAGACCGTGAAGTGCTTCAACTGTGGCAAAGAGGGACACATTGCCAAGA | |
| | ACTGCAGGGCTCCCAGGAAGAAAGGCTGCTGGAAGTGCGGAAAAGAAGGCCACCA | |
| | GATGAAGGACTGCACCGAGAGGCAGGCCAACTTCCTGGGCAAGATCTGGCCTAGC | |
| | AACAAGGGCAGGCCTGGCAACTTCCTGCAGAACAGACCCGAGCCCACCGCTCCTC | |
| | CCGAGGAAAGCTTCCGGTTTGGCGAGGAAACCACCACCCCTAGCCAGAAGCAGGA | |
| | ACCCATCGACAAAGAGATGTACCCTCTGGCCAGCCTGAAGAGCCTGTTCGGCAAC | |
| | GACCCCAGCAGCCAGATGGCTCCCATCAGCCCAATCGAGACAGTGCCTGTGAAGC | |
| | TGAAGCCTGGCATGGACGGACCCAGGGTGAAGCAGTGGCCTCTGACCGAGGAAAA | |
| | GATCAAAGCCCTGACAGCCATCTGCGAGGAAATGGAAAAAGAGGGCAAGATCACC | |
| | AAGATCGGACCCGAGAACCCCTACAACACCCCTGTGTTCGCCATCAAGAAGAAAG | |
| | ACAGCACCAAGTGGAGGAAACTGGTGGACTTCAGAGAGCTGAACAAGCGGACCCA | |
| | GGACTTCTGGGAGGTGCAGCTGGGCATCCCTCACCCTGCTGGCCTGAAGAAAAAG | |
| | AAAAGCGTGACCGTGCTGGCTGTGGGAGATGCCTACTTCAGCGTGCCTCTGGACG | |
| | AGGGCTTCCGGAAGTACACAGCCTTCACCATCCCCAGCACCAACAACGAGACACC | |
| | TGGCATCAGATACCAGTACAACGTGCTGCCTCAGGGCTGGAAAGGCAGCCCTGCC | |
| | ATCTTCCAGTGCAGCATGACCAGAATCCTGGAACCCTTCAGAGCCAAGAACCCTG | |
| | AGATCGTGATCTACCAGTATATGGCTGCCCTCTACGTGGGCAGCGACCTGGAAAT | |
| | CGGACAGCACAGAGCCAAAATCGAAGAACTCCGCGAGCACCTGCTGAAGTGGGGA | |
| | TTCACCACCCCTGACAAGAAGCACCAGAAAGAGCCTCCCTTCCTGTGGATGGGCT | |
| | ACGAGCTGCACCCTGACAAGTGGACCGTGCAGCCCATCCAGCTGCCAGAGAAGGA | |
| | CTCCTGGACCGTGAACGACATCCAGAAACTGGTCGGCAAGCTGAACTGGGCCAGC | |
| | CAGATCTACCCTGGCATCAAAGTCAGACAGCTGTGTAAGCTGCTGAGGGGAGCCA | |
| | AAGCACTGACCGACATCGTGCCTCTGACAGAAGAAGCCGAGCTGGAACTGGCCGA | |
| | GAACAGAGAGATCCTGAAAGAACCCGTGCACGGAGTGTACTACGACCCCTCCAAG | |
| | GACCTGATTGCCGAGATCCAGAAACAGGGACACGACCAGTGGACCTACCAGATCT | |
| | ATCAGGAACCTTTCAAGAACCTGAAAACAGGCAAGTACGCCAAGATGCGGACAGC | |
| | CCACACCAACGACGTGAAGCAGCTGACCGAAGCCGTGCAGAAAATCGCCATGGAA | |
| | AGCATCGTGATCTGGGGAAAGACACCCAAGTTCAGGCTGCCCATCCAGAAAGAGA | |
| | CATGGGAAACCTGGTGGACCGACTACTGGCAGGCCACCTGGATTCCCGAGTGGGA | |
| | GTTCGTGAACACCCCACCCCTGGTGAAGCTGTGGTATCAGCTGGAAAAGGACCCT | |
| | ATCGCTGGCGTGGAGACATTCTACGTGGCTGGAGCTGCCAACAGAGAGACAAAGC | |
| | TGGGCAAGGCTGGCTACGTGACCGACAGAGGCAGACAGAAAATCGTGAGCCTGAC | |
| | CGAAACCACCAACCAGAAAACAGCCCTGCAGGCCATCTATCTGGCACTGCAGGAC | |
| | AGCGGAAGCGAGGTGAACATCGTGACAGCCAGCCAGTATGCCCTGGGCATCATCC | |
| | AGGCCCAGCCTGACAAGAGCGAGAGCGAGCTGGTGAACCAGATCATCGAGCAGCT | |
| | GATCAAGAAAGAACGGGTGTACCTGAGCTGGGTGCCAGCCCACAAGGGCATCGGA | |
| | GGGAACGAGCAGGTGGACAAGCTGGTGTCCAGCGGAATCCGGAAGGTGCTGTTCC | |
| | TGGACGGCATCGATAAAGCCCAGGAAGAGCACGAGAAGTACCACAGCAATTGGAG | |
| | AGCCATGGCCAGCGACTTCAACCTGCCTCCCGTGGTGGCCAAAGAAATCGTGGCC | |
| | AGCTGCGACCAGTGCCAGCTGAAAGGCGAGGCCATGCACGGACAGGTGGACTGCT | |
| | CCCCTGGCATCTGGCAGCTGGCATGCACCCACCTGGAAGGCAAGATCATTCTGGT | |
| | GGCCGTGCACGTGGCCAGCGGATACATCGAAGCCGAAGTGATCCCTGCCGAGACA | |
| | GGGCAGGAAACAGCCTACTTCATCCTGAAGCTGGCTGGCAGATGGCCTGTGAAGG | |
| | TGATCCACACAGCCAACGGCAGCAACTTCACCTCTGCTGCCGTGAAGGCTGCCTG | |
| | TTGGTGGGCTGGCATTCAGCAGGAATTTGGCATCCCCTACAATCCCCAGTCTCAG | |
| | GGAGTGGTGGCCAGCATGAACAAAGAGCTGAAGAAGATCATCGGACAGGTCAGGG | |
| | ATCAGGCCGAGCACCTGAAAACTGCCGTCCAGATGGCCGTGTTCATCCACAACTT | |
| | CAAGCGGAAGGGAGGGATCGGAGGGTACTCTGCTGGCGAGCGGATCATCGACATC | |
| | ATTGCCACCGATATCCAGACCAAAGAGCTGCAGAAACAGATCATCAAGATCCAGA | |
| | ACTTCAGGGTGTACTACAGGGACAGCAGGGACCCCATCTGGAAGGGACCTGCCAA | |
| | GCTGCTGTGGAAAGGCGAAGGAGCCGTCGTCATCCAGGACAACAGCGACATCAAG | |
| | GTGGTGCCCAGACGGAAGGTGAAAATCATCAAGGACTACGGCAAACAGATGGCTG | |
| | GAGCCGACTGTGTCGCTGGCAGGCAGGACGAGGACTAATGA | |
| Mos1.Gag- | MGARASVLSGGELDRWEKIRLRPGGKKKYRLKHIVWASRELERFAVNPGLLETSE | 4 |
| Pol. | GCRQILGQLQPSLQTGSEELRSLYNTVATLYCVHQRIEIKDTKEALEKIEEEQNK | |
| (amino acid | SKKKAQQAAADTGNSSQVSQNYPIVQNIQGQMVHQAISPRTLNAWVKVVEEKAFS | |
| sequence) | PEVIPMFSALSEGATPQDLNTMLNTVGGHQAAMQMLKETINEEAAEWDRVHPVHA | |
| | GPIAPGQMREPRGSDIAGTTSTLQEQIGWMTNNPPIPVGEIYKRWIILGLNKIVR | |
| | MYSPVSILDIRQGPKEPFRDYVDRFYKTLRAEQASQDVKNWMTETLLVQNANPDC | |
| | KTILKALGPAATLEEMMTACQGVGGPGHKARVLAEAMSQVTNSATIMMQRGNFRN | |
| | QRKTVKCFNCGKEGHIAKNCRAPRKKGCWKCGKEGHQMKDCTERQANFLGKIWPS | |
| | NKGRPGNFLQNRPEPTAPPEESFRFGEETTTPSQKQEPIDKEMYPLASLKSLFGN | |
| | DPSSQMAPISPIETVPVKLKPGMDGPRVKQWPLTEEKIKALTAICEEMEKEGKIT | |
| | KIGPENPYNTPVFAIKKKDSTKWRKLVDFRELNKRTQDFWEVQLGIPHPAGLKKK | |
| | KSVTVLAVGDAYFSVPLDEGFRKYTAFTIPSTNNETPGIRYQYNVLPQGWKGSPA | |
| | IFQCSMTRILEPFRAKNPEIVIYQYMAALYVGSDLEIGQHRAKIEELREHLLKWG | |
| | FTTPDKKHQKEPPFLWMGYELHPDKWTVQPIQLPEKDSWTVNDIQKLVGKLNWAS | |
| | QIYPGIKVRQLCKLLRGAKALTDIVPLTEEAELELAENREILKEPVHGVYYDPSK | |
| | DLIAEIQKQGHDQWTYQIYQEPFKNLKTGKYAKMRTAHTNDVKQLTEAVQKIAME | |
| | SIVIWGKTPKFRLPIQKETWETWWTDYWQATWIPEWEFVNTPPLVKLWYQLEKDP | |
| | IAGVETFYVAGAANRETKLGKAGYVTDRGRQKIVSLTETTNQKTALQAIYLALQD | |
| | SGSEVNIVTASQYALGIIQAQPDKSESELVNQIIEQLIKKERVYLSWVPAHKGIG | |
| | GNEQVDKLVSSGIRKVLFLDGIDKAQEEHEKYHSNWRAMASDFNLPPVVAKEIVA | |
| | SCDQCQLKGEAMHGQVDCSPGIWQLACTHLEGKIILVAVHVASGYIEAEVIPAET | |
| | GQETAYFILKLAGRWPVKVIHTANGSNFTSAAVKAACWWAGIQQEFGIPYNPQSQ | |
| | GVVASMNKELKKIIGQVRDQAEHLKTAVQMAVFIHNFKRKGGIGGYSAGERIIDI | |
| | IATDIQTKELQKQIIKIQNFRVYYRDSRDPIWKGPAKLLWKGEGAVVIQDNSDIK | |
| | VVPRRKVKIIKDYGKQMAGADCVAGRQDED | |

### EXAMPLES

The following examples of the invention are to further illustrate the nature of the invention. It should be understood that the following examples do not limit the invention and that the scope of the invention is to be determined by the appended claims.

### Example 1: Mucosal Cellular Immune Responses Induced by Administration of a First and a Second Composition Comprising Adenoviral Vector Ad26 Vector Expressing a Fusion Protein of E6 and E7 of HPV16 in Mice

### Materials and Methods

Five days before immunization, animals were injected subcutaneous (SC) with Depo-provera (3 mg/mouse) to synchronize the menstrual cycle of the mice.

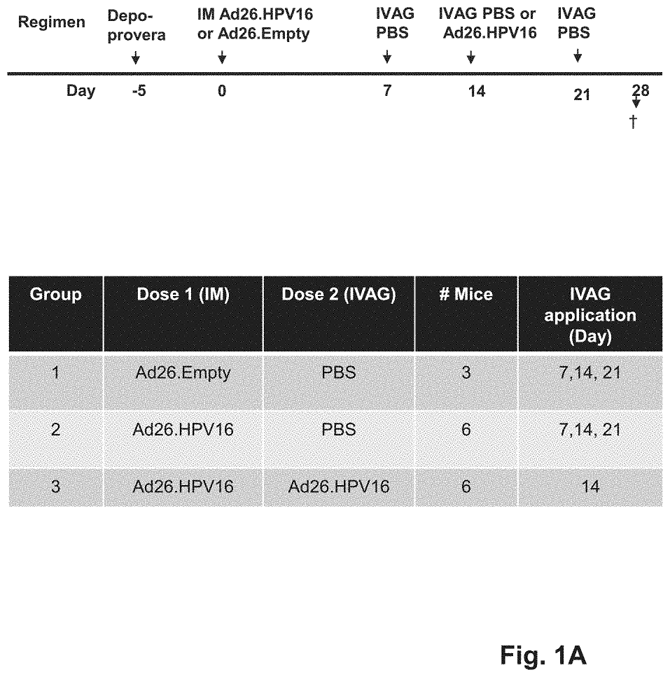

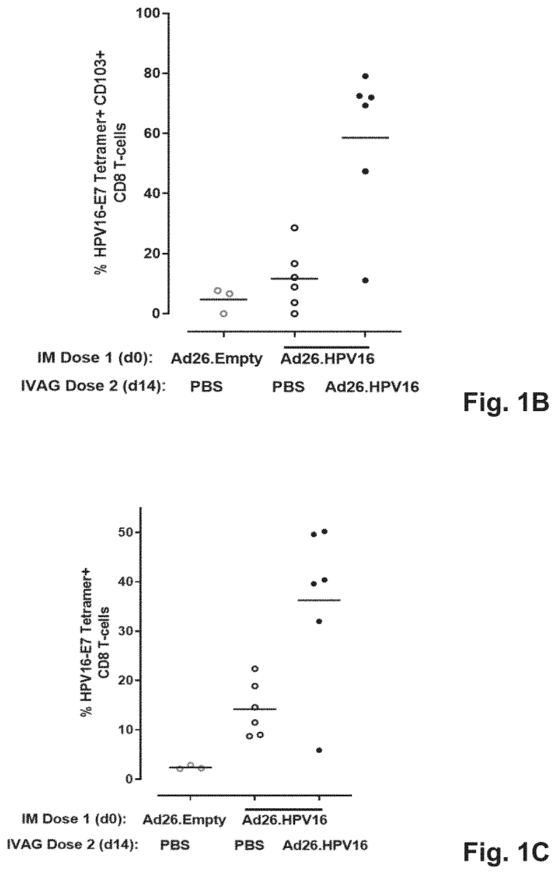

The Ad26 vectors tested expressed a fusion protein of E6 and E7 antigens of HPV16 (Ad26.HPV16, antigen sequence provided as SEQ ID NO: 2, nucleotide sequence provided as SEQ ID NO: 1). The vector was tested at a dose of 10¹⁰ virus particles (vp) administered intramuscularly (IM, group 2 and group 3), while the control mice (group 1) received 10¹⁰ of an Ad26 vector that did not express the transgene (Ad26.Empty). At day 7, day 14, and day 21, animals in group 1 and group 2 received PBS intravaginally (IVAG). Two weeks post 1st dosing, mice in group 3 received Ad26.HPV16 vector (10¹⁰ vp) that was resuspended in 4% Carboxymethylcellulose (a gel that helps the vaccine to remain localized within the vaginal tract) IVAG, or mice received PBS IVAG, and animals were sacrificed two weeks later (see FIG. 1A for schematic representation of the experimental set-up).

Immune responses against the HPV16-E7 antigen were measured using established immunological assays such as flow cytometry in combination with tetramer analysis.

### Results

A single IM immunization with Ad26.HPV16 induced some HPV16-E7-specific tissue resident memory T cell responses in the vaginal mucosa (FIG. 1B). In contrast, IM/IVAG homologous 2 dose Ad26.HPV16 immunization regimen enhanced the antigen-specific tissue resident memory CD8 T cells detected in the vaginal mucosa, resulting in more than 5-fold increase over that induced by a single IM immunization of Ad26.HPV16 (mean group response: IM Ad26.HPV16: 11.67%; IM/IVAG Ad26.HPV16: 58.57%). An ˜2.5-fold enhancement was also seen in the % of HPV16-E7-specific CD8 T-cells in the vaginal tract (FIG. 1C). The induction of CD103+ T-cells is a desired effect, as these are the cells needed to kill infected cells at the site of infection. For instance, CD103 is used as a surrogate marker to predict cervical T cell responses in the female genital tract during HIV infection (Kiravu et al, 2011, Clin Immunol. 141: 143-151), and CD103 was reported to be a promising marker for rapid assessment of tumor-reactive T cell infiltration of cervical cancers and a promising response biomarker for HPV E6/E7-targeted immunotherapy (Komdeur et al, 2017, Oncoimmunology, 2017, https://doi.org/10.1080/2162402X.2017.1338230). Thus, the regimen of the present invention, administration of an Ad26 vector that encodes an antigen of interest (rAd26) intramuscularly followed by administration of the same rAd26 intravaginally, results in strongly enhanced immune responses of the desired type. So far, such responses had only been shown with administrations of heterologous vectors at different time points, but surprisingly this now also appears possible by using the same Ad26 vector.

### Example 2: The Time Interval Between the IM First Dose and the IVAG Second Dose does not Impact the Quantity and Quality of the Induced Mucosal Cellular Immunity in Mice

### Materials and Methods

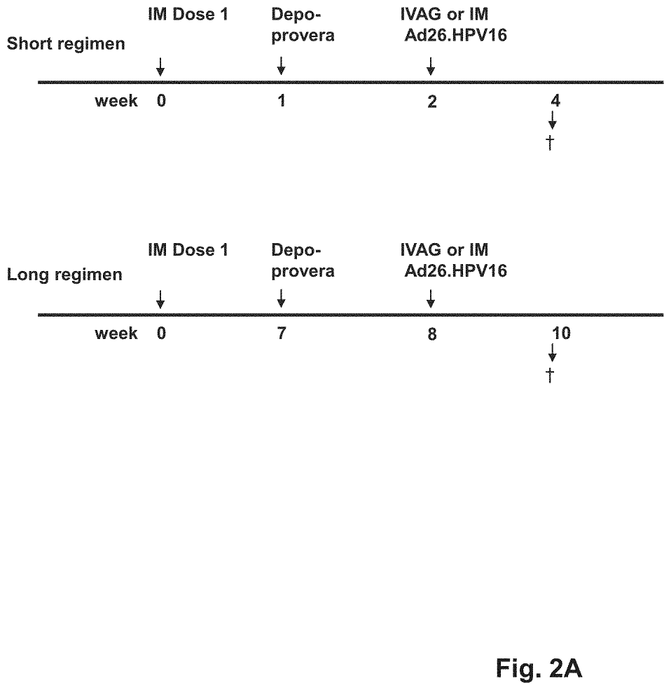

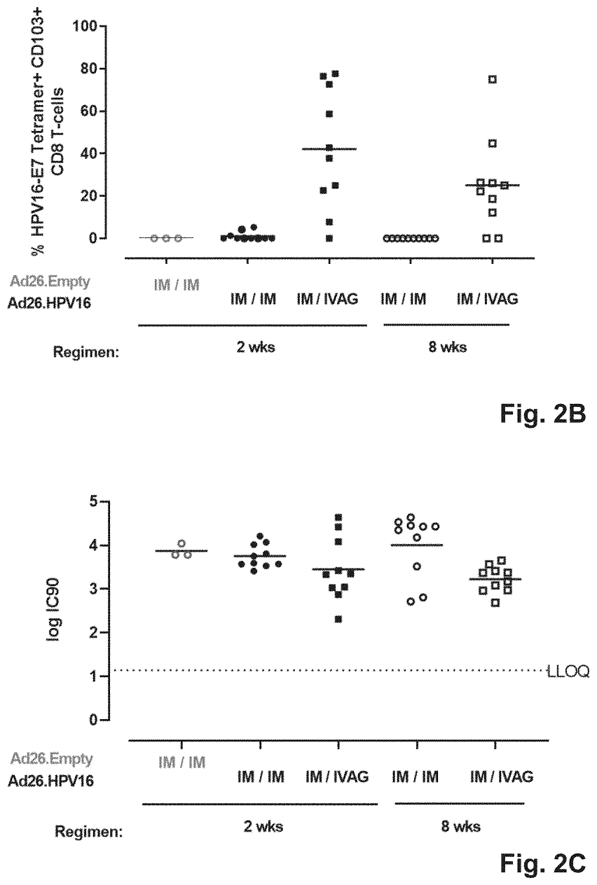

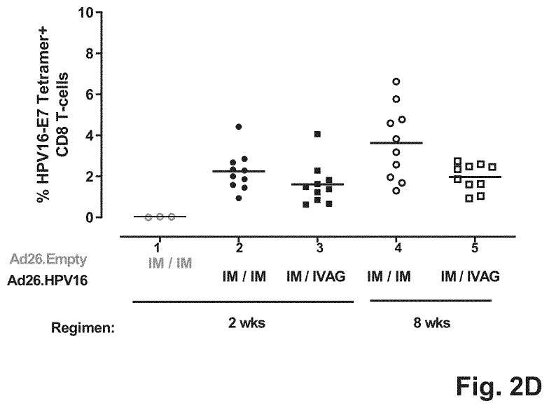

Mice received a first IM administration with Ad26.HPV16 or with an Ad26 vector not enclosing a transgene (Ad26.Empty) at 10¹⁰ VP. One week before the 2nd immunization, animals were injected SC with Depo-provera (3 mg/mouse) to synchronize the menstrual cycle of the mice. Subsequently, animals were dosed either at week 2 or at week 8 post first immunization with Ad26.HPV16, via the IM or IVAG route. Animals were sacrificed two weeks after the 2nd dosing (see FIG. 2A for schematic representation of the experimental set-up).

### Results

A significant higher induction of HPV16-E7 specific CD103+ tissue resident memory CD8 T-cells were detected in the cervicovaginal mucosal animals dosed via IM/IVAG, irrespective of the interval between the doses, compared to animals dosed via IM/IM with Ad26.HPV16 (FIG. 2B). The interval between the first dosing and the 2^{nd} dosing did not critically influence the induction of tissue resident memory T-cells specific for HPV16 in the cervicovaginal tissue (FIG. 2B).

There was a trend that IM/IVAG application induced slightly lower serum level of Ad26 neutralizing antibodies compared to IM/IM route of application (FIG. 2C). The systemic response to IM/IVAG and to IM/IM was also compared by measuring the % of HPV16-E7-specific CD8 T-cells in the spleen (FIG. 2D), no significant difference in systemic response was observed.

### Example 3: Comparison of HIV-Specific CD8 T-Cell Immune Response in Vaginal Mucosa after Intramuscular—Intravaginal 2 Dose Regimen with Ad26 Alone or in Combination with TLR Agonist Imiquimod Intravaginally

### Materials and Methods

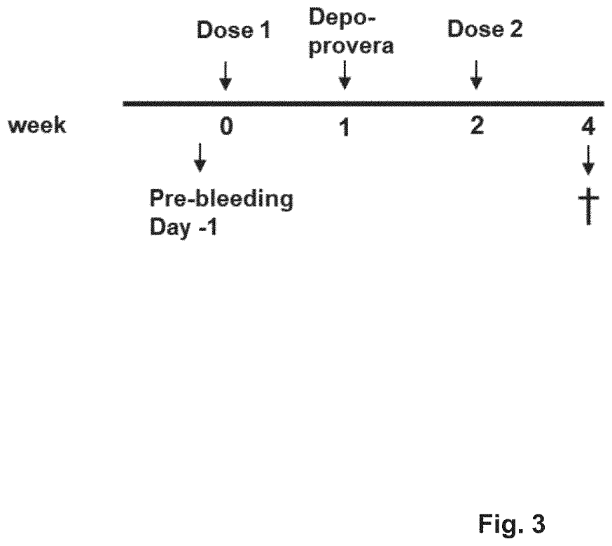

Mice receive a first IM administration with Ad26.Mos1.GagPol (antigen sequence provided as SEQ ID NO: 3, nucleotide sequence provided as SEQ ID NO: 4) or with an Ad26 vector not including a transgene (Ad26.Empty) at 10¹⁰ VP. One week before the 2nd immunization, animals are injected SC with Depo-provera (3 mg/mouse) to synchronize the menstrual cycle of the mice. Subsequently, animals are dosed at week 2 post first immunization with Ad26.Mos1.GagPol alone or with Ad26.Mos1.GagPol in combination with imiquimod gel via the IVAG route, or animals are injected intramuscularly with Ad26.Mos1.GagPol or with an Ad26 vector not including a transgene (Ad26.Empty) at 10¹⁰ VP. Animals are sacrificed two weeks after the 2nd dosing (see FIG. 3 for schematic representation of the experimental set-up). Induction of CD8 T-cells specific for the Mos1.GagPol insert is measured by various immunological techniques such as but not limited to flow cytometry analysis in combination with multimers to stain for HIV Gag specific CD8 T-cells in the vaginal tissue and in the spleen.

1. A method for inducing an immune response in a subject comprising:
intramuscularly administering to the subject a recombinant adenovirus of type Ad26 comprising a polynucleotide that encodes an antigen (rAd26); and
subsequently administering the rAd26 intravaginally to the subject.

2. The method according to claim 1, wherein the polynucleotide encodes a bacterial, parasitical, or viral antigen, or an antigenic fragment thereof.

3. The method according to claim 1, wherein the method is for inducing an immune response against an agent that causes a sexually transmitted disease (STD).

4. The method according to claim 3, wherein the sexually transmitted disease is selected from the group consisting of HIV infection, HPV infection, HSV infection, chlamydia, gonorrhea, syphilis and trichomoniasis.

5. The method according to claim 1, wherein the polynucleotide encodes an HPV E6 protein or antigenic part thereof and/or an HPV E7 protein or antigenic part thereof, preferably the polynucleotide encodes epitopes of both HPV E6 and E7 proteins.

6. The method according to claim 1, wherein the antigen comprises at least one of an HIV Env, Gag or Pol protein, or an antigenic part thereof.

7. The method according to claim 1, wherein the rAd26 is administered intravaginally between about one and ten weeks, preferably between about two and eight weeks, after the intramuscular administration.

8. A recombinant adenovirus of type Ad26 comprising a polynucleotide that encodes an antigen (rAd26) for use in inducing an immune response in a subject, wherein the rAd26 is for intramuscular administration, followed by intravaginal administration.

9. A method for treating/and or preventing a viral infection in a subject comprising administering to the subject a recombinant adenovirus of type Ad26 comprising a polynucleotide that encodes an antigen of the virus that caused the viral infection or to which prevention is desired (rAd26); wherein the administration comprises intramuscular administration of the rAd26 followed by intravaginal administration of the rAd26.

10. A kit of parts comprising:
a first composition comprising a recombinant adenovirus of type Ad26 comprising a polynucleotide that encodes an antigen (rAd26); and
a second composition comprising the rAd26;
wherein the first composition is for use in intramuscular administration and the second composition is for use in intravaginal administration.


SEQUENCE LISTING (4 sequences)

SEQ ID NO: 1 - DNA, 774 bp, Artificial Sequence
  HPV16.E6E7
atgcaccagaaacggaccgccatgttccaggacccccaggaacggcccagaaagctgccccagctgtgcaccgagctgcagaccaccatccacgacatcatcctggaatgcgtgtactgcaagcagcagctcctgcggagagaggtgtacgacttcgccttccgggacctgtgtatcgtgtaccgggacggcaacccctacgccgtgtgcgacaagtgcctgaagttctacagcaagatcagcgagtaccggcactactgctacagcctgtacggcaccaccctggaacagcagtacaacaagcccctgtgcgacctgctgatccggtgcatcaactgccagaaacccctgtgccccgaggaaaagcagcggcacctggacaagaagcagcggttccacaacatccggggcagatggacaggccggtgcatgagctgctgcagaagcagccggaccagacgggaaacccagctgatgcacggcgacacccccaccctgcacgagtacatgctggacctgcagcccgagacaaccgacctgtactgctacgagcagctgaacgacagcagcgaggaagaggacgagatcgacggccctgctggacaggccgagcctgatagagcccactacaatatcgtgaccttctgctgcaagtgcgacagcaccctgcggctgtgcgtgcagagcacccacgtggacatccggaccctggaagatctgctgatgggcaccctgggcatcgtgtgccccatctgctcccagaaaccctgataa

SEQ ID NO: 2 - protein, 256 aa, Artificial Sequence
  .HPV16.E6E7
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - DNA, 4056 bp, Artificial Sequence
  Mos1.gag-pol
atgggagccagagccagcgtgctgtccggaggggagctggaccgctgggagaagatcaggctgaggcctggagggaagaagaagtacaggctgaagcacatcgtgtgggccagcagagagctggaacggtttgccgtgaaccctggcctgctggaaaccagcgagggctgtaggcagattctgggacagctgcagcccagcctgcagacaggcagcgaggaactgcggagcctgtacaacaccgtggccaccctgtactgcgtgcaccagcggatcgagatcaaggacaccaaagaagccctggaaaagatcgaggaagagcagaacaagagcaagaagaaagcccagcaggctgccgctgacacaggcaacagcagccaggtgtcccagaactaccccatcgtgcagaacatccagggacagatggtgcaccaggccatcagccctcggaccctgaacgcctgggtgaaggtggtggaggaaaaggccttcagccctgaggtgatccccatgttctctgccctgagcgagggagccacaccccaggacctgaacaccatgctgaacaccgtgggagggcaccaggctgccatgcagatgctgaaagagacaatcaacgaggaagctgccgagtgggacagggtccacccagtgcacgctggacctatcgctcctggccagatgagagagcccagaggcagcgatattgctggcaccacctccacactgcaggaacagatcggctggatgaccaacaaccctcccatccctgtgggagagatctacaagcggtggatcattctgggactgaacaagatcgtgcggatgtacagccctgtgagcatcctggacatcaggcagggacccaaagagcccttcagggactacgtggaccggttctacaagaccctgagagccgagcaggccagccaggacgtgaagaactggatgaccgagacactgctggtgcagaacgccaaccctgactgcaagaccatcctgaaagccctgggacctgctgccaccctggaagagatgatgacagcctgccagggagtgggaggacctggccacaaggccagggtgctggccgaggccatgagccaggtgaccaactctgccaccatcatgatgcagagaggcaacttccggaaccagagaaagaccgtgaagtgcttcaactgtggcaaagagggacacattgccaagaactgcagggctcccaggaagaaaggctgctggaagtgcggaaaagaaggccaccagatgaaggactgcaccgagaggcaggccaacttcctgggcaagatctggcctagcaacaagggcaggcctggcaacttcctgcagaacagacccgagcccaccgctcctcccgaggaaagcttccggtttggcgaggaaaccaccacccctagccagaagcaggaacccatcgacaaagagatgtaccctctggccagcctgaagagcctgttcggcaacgaccccagcagccagatggctcccatcagcccaatcgagacagtgcctgtgaagctgaagcctggcatggacggacccagggtgaagcagtggcctctgaccgaggaaaagatcaaagccctgacagccatctgcgaggaaatggaaaaagagggcaagatcaccaagatcggacccgagaacccctacaacacccctgtgttcgccatcaagaagaaagacagcaccaagtggaggaaactggtggacttcagagagctgaacaagcggacccaggacttctgggaggtgcagctgggcatccctcaccctgctggcctgaagaaaaagaaaagcgtgaccgtgctggctgtgggagatgcctacttcagcgtgcctctggacgagggcttccggaagtacacagccttcaccatccccagcaccaacaacgagacacctggcatcagataccagtacaacgtgctgcctcagggctggaaaggcagccctgccatcttccagtgcagcatgaccagaatcctggaacccttcagagccaagaaccctgagatcgtgatctaccagtatatggctgccctctacgtgggcagcgacctggaaatcggacagcacagagccaaaatcgaagaactccgcgagcacctgctgaagtggggattcaccacccctgacaagaagcaccagaaagagcctcccttcctgtggatgggctacgagctgcaccctgacaagtggaccgtgcagcccatccagctgccagagaaggactcctggaccgtgaacgacatccagaaactggtcggcaagctgaactgggccagccagatctaccctggcatcaaagtcagacagctgtgtaagctgctgaggggagccaaagcactgaccgacatcgtgcctctgacagaagaagccgagctggaactggccgagaacagagagatcctgaaagaacccgtgcacggagtgtactacgacccctccaaggacctgattgccgagatccagaaacagggacacgaccagtggacctaccagatctatcaggaacctttcaagaacctgaaaacaggcaagtacgccaagatgcggacagcccacaccaacgacgtgaagcagctgaccgaagccgtgcagaaaatcgccatggaaagcatcgtgatctggggaaagacacccaagttcaggctgcccatccagaaagagacatgggaaacctggtggaccgactactggcaggccacctggattcccgagtgggagttcgtgaacaccccacccctggtgaagctgtggtatcagctggaaaaggaccctatcgctggcgtggagacattctacgtggctggagctgccaacagagagacaaagctgggcaaggctggctacgtgaccgacagaggcagacagaaaatcgtgagcctgaccgaaaccaccaaccagaaaacagccctgcaggccatctatctggcactgcaggacagcggaagcgaggtgaacatcgtgacagccagccagtatgccctgggcatcatccaggcccagcctgacaagagcgagagcgagctggtgaaccagatcatcgagcagctgatcaagaaagaacgggtgtacctgagctgggtgccagcccacaagggcatcggagggaacgagcaggtggacaagctggtgtccagcggaatccggaaggtgctgttcctggacggcatcgataaagcccaggaagagcacgagaagtaccacagcaattggagagccatggccagcgacttcaacctgcctcccgtggtggccaaagaaatcgtggccagctgcgaccagtgccagctgaaaggcgaggccatgcacggacaggtggactgctcccctggcatctggcagctggcatgcacccacctggaaggcaagatcattctggtggccgtgcacgtggccagcggatacatcgaagccgaagtgatccctgccgagacagggcaggaaacagcctacttcatcctgaagctggctggcagatggcctgtgaaggtgatccacacagccaacggcagcaacttcacctctgctgccgtgaaggctgcctgttggtgggctggcattcagcaggaatttggcatcccctacaatccccagtctcagggagtggtggccagcatgaacaaagagctgaagaagatcatcggacaggtcagggatcaggccgagcacctgaaaactgccgtccagatggccgtgttcatccacaacttcaagcggaagggagggatcggagggtactctgctggcgagcggatcatcgacatcattgccaccgatatccagaccaaagagctgcagaaacagatcatcaagatccagaacttcagggtgtactacagggacagcagggaccccatctggaagggacctgccaagctgctgtggaaaggcgaaggagccgtcgtcatccaggacaacagcgacatcaaggtggtgcccagacggaaggtgaaaatcatcaaggactacggcaaacagatggctggagccgactgtgtcgctggcaggcaggacgaggactaatga

SEQ ID NO: 4 - protein, 1350 aa, Artificial Sequence
  Mos1.Gag-Pol.v3
MGARASVLSGGELDRWEKIRLRPGGKKKYRLKHIVWASRELERFAVNPGLLETSEGCRQILGQLQPSLQTGSEELRSLYNTVATLYCVHQRIEIKDTKEALEKIEEEQNKSKKKAQQAAADTGNSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPVSILDIRQGPKEPFRDYVDRFYKTLRAEQASQDVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSNKGRPGNFLQNRPEPTAPPEESFRFGEETTTPSQKQEPIDKEMYPLASLKSLFGNDPSSQMAPISPIETVPVKLKPGMDGPRVKQWPLTEEKIKALTAICEEMEKEGKITKIGPENPYNTPVFAIKKKDSTKWRKLVDFRELNKRTQDFWEVQLGIPHPAGLKKKKSVTVLAVGDAYFSVPLDEGFRKYTAFTIPSTNNETPGIRYQYNVLPQGWKGSPAIFQCSMTRILEPFRAKNPEIVIYQYMAALYVGSDLEIGQHRAKIEELREHLLKWGFTTPDKKHQKEPPFLWMGYELHPDKWTVQPIQLPEKDSWTVNDIQKLVGKLNWASQIYPGIKVRQLCKLLRGAKALTDIVPLTEEAELELAENREILKEPVHGVYYDPSKDLIAEIQKQGHDQWTYQIYQEPFKNLKTGKYAKMRTAHTNDVKQLTEAVQKIAMESIVIWGKTPKFRLPIQKETWETWWTDYWQATWIPEWEFVNTPPLVKLWYQLEKDPIAGVETFYVAGAANRETKLGKAGYVTDRGRQKIVSLTETTNQKTALQAIYLALQDSGSEVNIVTASQYALGIIQAQPDKSESELVNQIIEQLIKKERVYLSWVPAHKGIGGNEQVDKLVSSGIRKVLFLDGIDKAQEEHEKYHSNWRAMASDFNLPPVVAKEIVASCDQCQLKGEAMHGQVDCSPGIWQLACTHLEGKIILVAVHVASGYIEAEVIPAETGQETAYFILKLAGRWPVKVIHTANGSNFTSAAVKAACWWAGIQQEFGIPYNPQSQGVVASMNKELKKIIGQVRDQAEHLKTAVQMAVFIHNFKRKGGIGGYSAGERIIDIIATDIQTKELQKQIIKIQNFRVYYRDSRDPIWKGPAKLLWKGEGAVVIQDNSDIKVVPRRKVKIIKDYGKQMAGADCVAGRQDED